(19) United States
(12) Patent Application Publication
(10) Pub. No.: US 2003/0059823 A1
(43) Pub. Date: Mar. 27, 2003

(54) Hybridization apparatus and method for detecting nucleic acid in sample using the same

(75) Inventors: Tadashi Matsunaga, Koganei-shi (JP); Kiyoshi Yoda, Chofu-shi (JP); Yuji Udagawa, Chofu-shi (JP); Etsuo Nemoto, Chofu-shi (JP); Kohei Maruyama, Chofu-shi (JP)

Correspondence Address: MERCHANT & GOULD PC, P.O. BOX 2903, MINNEAPOLIS, MN, 55402-0903, US

(73) Assignee: Juki Corporation, Tokyo (JP)

(21) Appl. No.: 10/246,959

(22) Filed: Sep. 18, 2002

(30) Foreign Application Priority Data
Sep. 21, 2001 (JP) 2001-289938
Sep. 21, 2001 (JP) 2001-289939
Sep. 21, 2001 (JP) 2001-289940

(51) Int. Cl. C12Q 1/68; C12M 1/34

(52) U.S. Cl. 435/6; 435/287.2

(57) ABSTRACT

A hybridization apparatus includes at least (A) a reaction station having a reaction vessel holder, a heating-cooling device, and a magnetic force controller, (B) a tip rack/waste solution station having a tip rack and a waste solution reservoir, (C) a washing solution station having a washing solution reservoir and a heating-cooling device, and (D) a head station having an arm unit movable in X-Z directions, the arm unit including a tip setting mechanism having a 1o plurality of tip nozzles for respective tips to be attached to or detached from, and a mechanism for the attached tips to suck and inject treatment solution.

### BACKGROUND OF THE INVENTION

1. Field of the Invention

The invention relates to a hybridization apparatus that automatically performs hybridization using nucleic acid probes and a method for detecting a nucleic acid in a sample using this apparatus.

2. Description of the Related Art

Identification of infectious disease-causing germs, preclinical diagnosis of infectious diseases, and further diagnosis of genetic disorder have been realized recently, by analyzing a specific base sequence in a sample based on nucleic acid hybridization technique with the use of DNA probes.

The nucleic acid hybridization method includes, for example, dot hybridization and sandwich hybridization. The dot hybridization method includes the steps of immobilizing a single-stranded nucleic acid denatured from a sample onto a solid phase carrier, reacting a nucleic acid labeled with radioisotope or fluorescent dyes on the carrier to form a hybrid nucleic acid bound with the immobilized one, removing the free labeled nucleic acid not bound therewith, and measuring the intensity of radiation or luminescence emitted from the solid phase carrier. The sandwich hybridization method utilizes at least two nucleic acid fragments relating to a target nucleic acid to be discriminated.

The work to measure a sample nucleic acid using such a hybridization method requires simple repeated operations due to lots of samples, a large installation area of equipments for independent processes, long tact time for reaction due to reaction temperature control, and further precise handling of a trace of the sample. There has been therefore a great demand for mechanically automated system for hybridization process.

There has been known some apparatus for automatically detecting a nucleic acid using hybridization process. For example, U.S. Pat. No. 5,538,849 discloses an apparatus for an automated assay of a DNA probe, which includes a DNA sample-reagent unit, a sample-reagent dispense unit, a reaction vessel transport unit, a hybridization unit, a B/F separation unit, and a light measurement unit. However, this apparatus has plural vessels of samples and reagents, and does not simultaneously treat the plural samples, resulting in longer detection tact time. Thus, this apparatus has not been sufficient from the viewpoint of a small-sized apparatus and efficient hybridization process.

### SUMMARY OF THE INVENTION

Accordingly, an object of the invention is to provide a hybridization apparatus that can perform hybridization efficiently in short tact time.

Another object of the invention is to provide the apparatus that can obtain precise detection result by controlling reaction temperature with smaller experimental space.

As a result of an intensive study in automated hybridization of a nucleic acid by the inventors with the foregoing in mind, it has been found that employment of heating-cooling devices as well as a magnetic force controller allows to automate the hybridization process with simple operations and to reduce the entire size of the apparatus.

The invention provides for an automatic nucleic acid hybridization apparatus. The apparatus includes at least (A) a denature station having a reaction vessel holder and a heating-cooling device, (B) an annealing station having a reaction vessel holder and a heating-cooling device, (C) a magnetic separation station having a reaction vessel holder and a magnetic force controller, (D) a tip rack storing station having a tip rack, (E) a washing solution station having a washing solution reservoir, (F) a waste solution station having a waste solution reservoir, and (G) a head station having an arm unit movable in X-Z directions, the arm unit including a tip setting mechanism having a plurality of tip nozzles for respective tips to be attached to or detached from, a mechanism for the attached tips to suck and inject treatment solution, and a robot-hand mechanism capable of holding and releasing a reaction vessel.

The invention also provides a method for detecting a nucleic acid in a sample using the above hybridization apparatus. The method automatically executes the following steps (1)-(10) and further (11)-(15) if necessary, and thereafter measures the amount of labeled nucleic acid in the reaction vessel:

(1) Setting on the denature station the reaction vessel in which either a nucleic acid probe immobilized on magnetic particles, a labeled probe and a sample nucleic acid, or a nucleic acid probe immobilized on magnetic particles and a labeled sample nucleic acid are injected and mixed, setting the temperature inside the vessel to a denaturing temperature of the nucleic acid by the heating-cooling device, and making the sample nucleic acid single-stranded with the temperature kept for a certain period of time;

(2) Transporting the reaction vessel on the denature station to the annealing station with actuation of the arm unit;

(3) Annealing the nucleic acid by setting the temperature inside the vessel to an annealing temperature by the heating-cooling device with the temperature kept for a certain period of time;

(4) Transporting the reaction vessel from the annealing station to the magnetic separation station by the arm unit;

(5) Biasing the nucleic acid bound with the magnetic particles in the vessel with the magnetic force controller energized;

(6) Transporting the arm unit to the tip rack storing station, and attaching tips to respective tip nozzles;

(7) Transporting the arm unit to the magnetic separation station, and sucking supernatant solution in the reaction vessel by the tip nozzles;

(8) Transporting the arm unit to the waste solution station, and discharging the sucked supernatant solution into the waste solution reservoir;

(9) Transporting the arm unit to the washing solution station, sucking the washing solution from the washing solution reservoir, and dispensing the washing solution into the reaction vessel on the magnetic separation station;

(10) Repeating the washing operation specified in steps (7)-(9) by given times;

(11) After finishing steps (7)-(8), transporting the arm unit to a first reagent station, sucking a marking reagent from a reagent reservoir, dispensing the sucked reagent into the reaction vessel on the magnetic separation station, and leaving still for a certain period of time;

(12) Sucking the supernatant solution in the reaction vessel by the tip nozzles, transporting the arm unit to the waste solution station, and discharging the sucked supernatant in the tip nozzles into the waste solution reservoir;

(13) Transporting the arm unit to a second washing solution station, sucking washing solution from a second washing solution reservoir, dispensing it into the reaction vessel on the magnetic separation station, and leaving still for a certain period of time;

(14) Repeating the washing operation specified in steps (12)-(13) by given times, and executing step (12); and

(15) Transporting the arm unit to a second reagent station, sucking a color developing agent from a reagent reservoir, and dispensing it into the reaction vessel on the magnetic separation station.

The invention provides another structure of automatic nucleic acid hybridization apparatus. The apparatus includes at least (A′) a reaction station having a reaction vessel holder, a heating-cooling device, and a magnetic force controller, (B′) a tip rack-waste solution station having a tip rack and a waste solution reservoir, (C′) a washing solution station having a washing solution reservoir and a heating-cooling device, and (D′) a head station having an arm unit movable in X-Z directions, the arm unit including a tip setting mechanism having a plurality of tip nozzles for respective tips to be attached to or detached from, and a mechanism for the attached tips to suck and inject treatment solution.

The invention also provides another method for detecting a nucleic acid in a sample using another hybridization apparatus. The method automatically executes the following steps (1′)-(8′), and thereafter measures the amount of labeled nucleic acid in the reaction vessel:

(1′) Setting on the reaction station the reaction vessel in which a nucleic acid probe immobilized onto magnetic particles and a labeled sample nucleic acid are injected and mixed, setting the temperature inside the vessel to a denaturing temperature of the nucleic acid by the heating-cooling device, and making the sample nucleic acid single-stranded with the temperature kept for a certain period of time;

(2′) Changing the temperature inside the vessel to an annealing temperature to anneal the nucleic acid with the temperature kept for a certain period of time;

(3′) Biasing the nucleic acid bound with the magnetic particles in the vessel with the magnetic force controller energized (B/F separation);

(4′) Transporting the arm unit to the tip rack-waste solution station, and attaching tips to respective tip nozzles;

(5′) Transporting the arm unit to the reaction station, and sucking supernatant solution in the reaction vessel by the tip nozzles;

(6′) Transporting the arm unit to the tip rack-waste solution station, and discharging the sucked supernatant solution into the waste solution reservoir;

(7′) Transporting the arm unit to the washing solution station, sucking from the washing solution reservoir the washing solution previously adjusted to the annealing temperature by the heating-cooling device with immersion of the nozzles in the washing solution for a certain period of time, and injecting the washing solution into the reaction vessel of the reaction station; and

(8′) Repeating the washing operation specified in steps (5′)-(7′) by given times.

### BRIEF DESCRIPTION OF THE DRAWINGS

FIG. 1 is a schematic diagram showing the internal structure of an automatic nucleic acid hybridization apparatus according to a first embodiment of the invention.

FIG. 2 is a side view showing a denature station 1 or an annealing station 2 of the apparatus according to the first embodiment.

FIG. 3 is a side view showing a magnetic separation station 3 of the apparatus according to the first embodiment.

FIG. 4 is a schematic diagram showing the internal structure of an automatic nucleic acid hybridization apparatus according to a second embodiment of the invention.

FIG. 5 is a side view showing a reaction station of the apparatus according to the second embodiment.

FIG. 6 illustrates magnetic force control based on parallel translation of a magnet.

FIG. 7 illustrates magnetic force control based on rotation of a magnet.

FIG. 8 illustrates magnetic force control based on 180° rotation of a magnet.

FIGS. 9(A) to 9(C) are a series of illustrations showing a principle of magnetic particle movement due to ON/OFF of magnetic force given from under a reaction vessel.

### DETAILED DESCRIPTION OF THE PREFERRED EMBODIMENTS

<First Embodiment>

A first embodiment of the invention will now be explained with reference to the accompanying drawings.

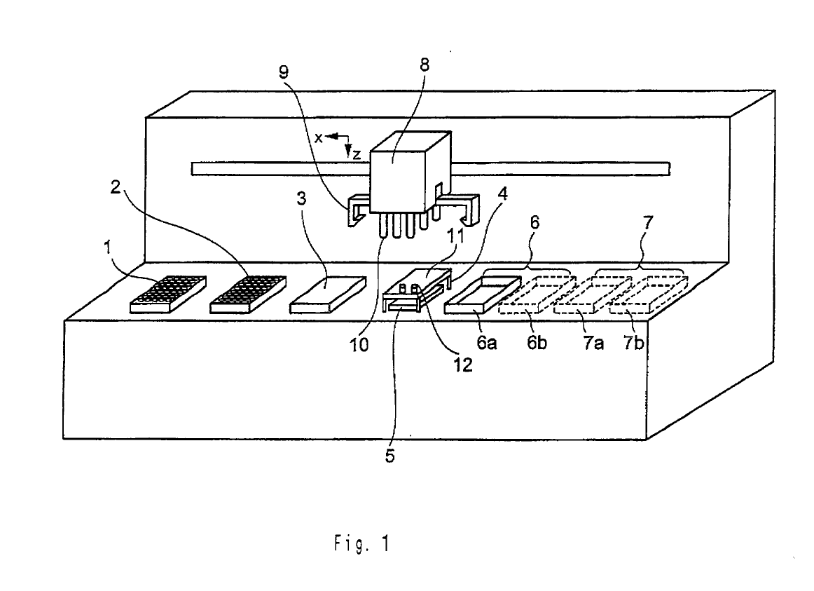

FIG. 1 is a schematic diagram showing the internal structure of an automatic nucleic acid hybridization apparatus according to the first embodiment of the invention. Numeral 1 denotes a denature station for making a sample nucleic acid single-stranded (denatured), and 2 an annealing station for hybridizing (annealing) the single-stranded sample nucleic acid with specific nucleic acid probes. Each of the denature station and the annealing station includes a reaction vessel holder for holding a reaction vessel, and a heating-cooling device for adjusting a denaturing or annealing temperature.

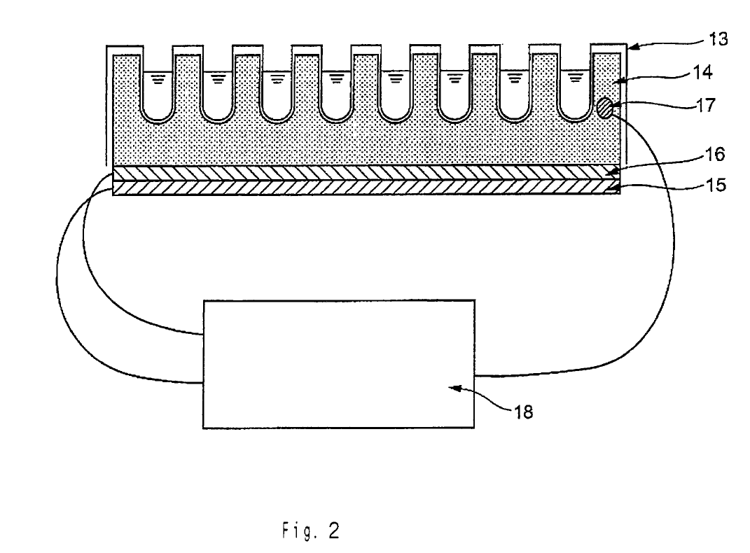

FIG. 2 is a side view showing the denature station 1 and the annealing station 2. Numeral 13 denotes a reaction vessel for executing therein the denaturing process and the annealing process, respectively. The shape and material of the vessel 13 are not particularly limited as long as they are suitable for the hybridization of nucleic acid, but it is preferable to use a general-purpose plastic 24-96 well microplate on which plural samples are to be simultaneously tested.

The heating-cooling device includes a heater 15 for heating the reaction vessel holder a 14, a chiller 16 for cooling it, a sensor 17 for detecting the temperature, and a temperature controller 18 for controlling the temperature. Employed as the vessel holder 14 is a metal plate shaped to be in close contact with the surface of the vessel 13 to be used.

The temperature controller on the denature station 1 sets the inside temperature of the vessel to 95° C. (may be set to lower than this in case of shorter length of a sample nucleic acid) for accelerating denature reaction. The controller 18 on the annealing station 2 sets to 40-70° C. for hybridization (annealing). Reaction time is also controlled with a timer function of the controller 18.

After the denature reaction in the denature station 1, the arm unit of the head station 8 transports the vessel 13 held on the station 1 onto the vessel holder 14 of the annealing station 2.

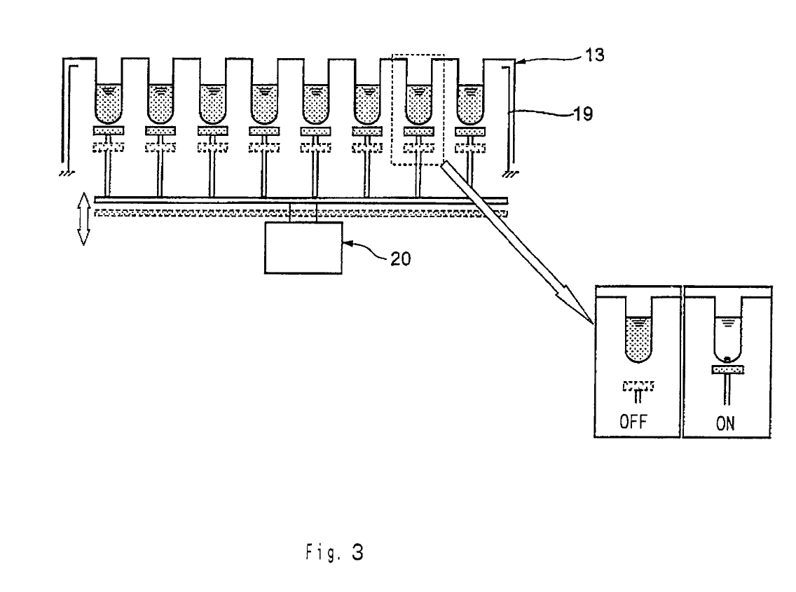

FIG. 3 is a side view showing a magnetic separation station 3. The magnetic separation station 3 includes a reaction vessel holder b 19 for fixing the reaction vessel, and a magnetic force controller 20.

The magnetic separation station 3 is a stage for B/F separation in the apparatus. The magnetic force controller 20 applies to each reaction vessel corresponding magnetic field to act on magnetic particles in the vessel for B/F separation.

The controller 20 is so disposed as to collect the magnetic particles into the bottom only of the vessel and to make them immovable with the application of the magnetic field. Placement of a magnetic force source just under the reaction vessel allows the magnetic particles to be collected to narrow area of the vessel bottom. It is not preferable for collection of the particles that a magnetic force source is moved closer to the side of the vessel, because the magnetic particles are elongated in an up-and-down direction along magnetic flux, which sometimes causes lots of magnetic particles to be adsorbed on the inner surface of the vessel if it is made of plastic.

In practice, the magnetic force can be controlled by placing a magnetic force source under the reaction vessel with the source turned ON and OFF. For instance, the magnetic force acting on the particles is increased with the magnetic source getting close to the vessel bottom, and decreased with the source moved apart downward or sideward.

Permanent magnets, electromagnets or the like can be used as the magnetic force source, out of which permanent magnets are preferably employed considering its flux density and volume. The permanent magnets are preferably arrayed corresponding to respective wells on the 96-well microplate with their magnetic poles N and S alternately arranged next to each other. This arrangement prevents disturbance of flux acting on each reaction vessel (well), thereby collecting the magnetic particles effectively.

B/F separation method will be explained in more detail.

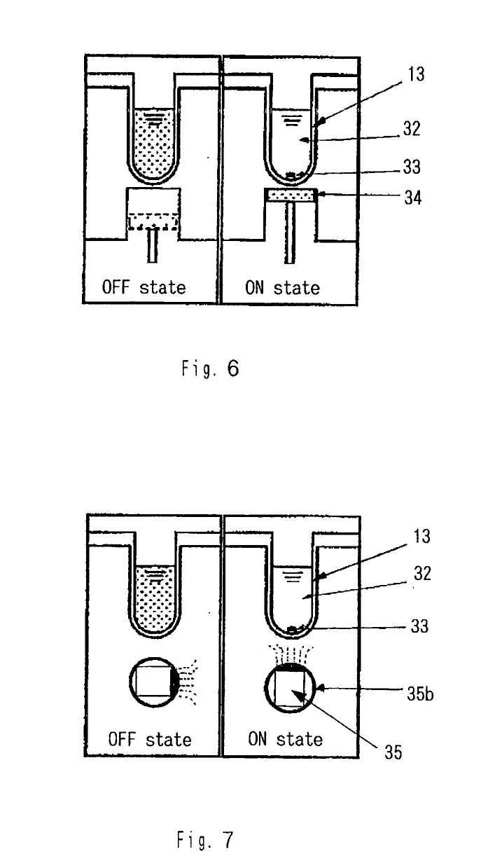

There may be various means of turning ON and OFF the magnetic force produced under the reaction vessel; for example, by (1) vertically moving permanent magnets 34 disposed under the vessel (FIG. 6), (2) horizontally moving permanent magnets disposed under the vessel by a half pitch, or complete disposition, (3) rotating a ferromagnetism having permanent magnets disposed under the vessel, for example, the ferromagnetism being a ferromagnetic rod 35b with permanent magnets 35 embedded, the rod being rotated to change the direction of magnetic flux (FIG. 7), or a ferromagnetic rod (magnet holder 36b) with each one end of magnet 36 protruded (FIG. 8), (4) horizontally moving in and out a shielding member against permanent magnets, (5) round-shaped electromagnets, (6) horseshoe electromagnets, and (7) electromagnets without core.

Means (1), (2) and (3) out of these ON-OFF means are preferable from their simplicity. The means shown in FIG. 8 can effectively direct the magnetic force to the vessel because the magnetic flux extends toward end surface direction. Each insertion hole for the magnet 36 on the holder 36b does not pass through the magnet holder 36b, and has enough thickness for the flux not to leak. With this structure, magnetic flux at holding end passes inside the magnet holder 36b without affecting outside, to thereby completely shut the magnetic flux from the vessel 13 at its OFF position. In B/F separation using this means, it is preferable to rotate the magnets by 180±30 degrees for switching between ON (right side illustration in FIG. 8) and OFF states.

The B/F separation of the invention is performed by given times of repetition of the following steps (1) and (2): (1) sucking and eliminating the supernatant solution 32 with the magnetic force controlled so that the magnetic particles 33 are collected into the bottom only of the vessel 13 and made immovable, and (2) injecting washing solution into the vessel under the immovable state of the particles, or after making the particles movable by controlling the magnetic force. Following step (3) may be further applied: (3) repeating ON-OFF of the magnetic force to move the magnetic particles in the washing solution before sucking and eliminating the supernatant solution.

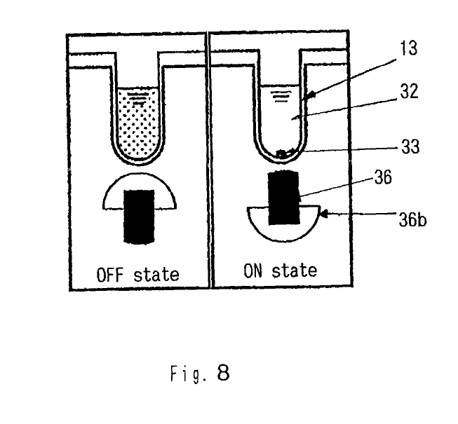

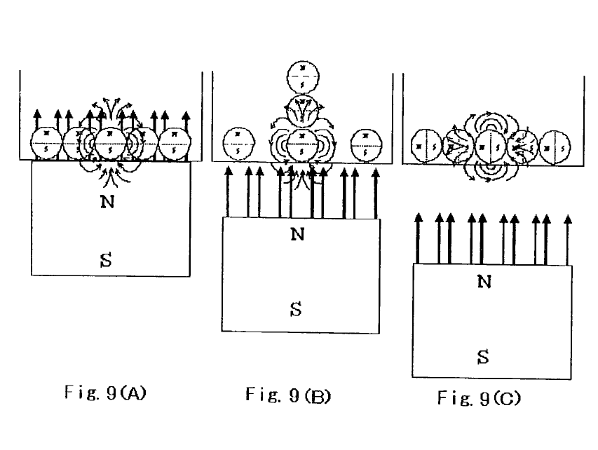

A principle of moving the magnetic particles in the washing solution with repetition of turning ON-OFF of the magnetic field given from under the reaction vessel will be explained with reference to FIGS. 9(A) to 9(C). When the magnetic force is not given to the particle as shown in FIG. 9(C), the magnetic particles, each having self-magnetization, lie in the solution such that N-pole of each particle is in contact with S-pole of other particle. When the magnetic force is given from under the vessel with the N-pole directed upward beyond the mutual magnetism on the particles as shown in FIG. 9(A), the particles rotate at 90° with each S-pole directed downward. Same poles face to each other with repulsion. When the mutual magnetism of the particles becomes over the given magnetic force with its gradual reduction, some particles align in headstand with the mutual magnetism as shown in FIG. 9(B). Thus, if the magnetic force is forcibly controlled with repeated cycles of strong, weak and OFF, the particles move about in the solution, which causes to release nonspecifically adsorbed substance from the surface of the particle.

If the particles are collected and made immovable at the vessel bottom only, the particles are hardly adsorbed on the surface of tip or tip nozzle in B/F separation and washing process, even if bacterial magnetic particles, which are easily adsorbed on plastic, are used as magnetic particles.

In B/F separation and washing process, the particles are held immovable at the bottom of the vessel even when the magnetic force is turned OFF, but disperses a little when the washing solution is injected. Although it is possible to suck and discharge the solution under the immovable state with the force turned ON again, it may also be possible to wash the particles with the force kept ON without turning OFF, which makes the washing process speed higher.

Repeated ON-OFF cycles of the magnetic force allows to reduce the repeating number of washing steps, which reduces possibility of erroneously sucking the magnetic particles caused by the sucking operation of the supernatant solution.

When the magnetic force is turned OFF after the washing process, B/F separated solid carriers to be measured remain in the reaction vessel. This vessel itself after B/F separation is submitted to a measurement station (not shown) to measure the luminescence or fluorescence emitted from the particles collected at the bottom.

A description will be given of the magnetic particle to be used in the nucleic acid hybridization in the invention.

The magnetic particles are not particularly limited as long as they are insoluble and acid in aqueous solution. The particles are, for example, FeO (about 200 Å in diameter) covered with FeO, γ-FeO, Co-γ-FeO, (NiCuZn)O.FeO, (CuZn)O.FeO, (MnZn)O.FeO, (NiZn)O.FeO, SrO.6FeO, BaO.6FeO, SiO₂ (refer to Enzyme Microb. Technol., vol. 2, p.2-10 (1980)), composite particles composed of ferrite and various high-polymers (Nylon, polyacrylamide, polystyrene, etc.), and bacterial magnetic particles that are formed inside magnetic bacteria.

The magnetic bacterium, which was discovered in the 1970s in America, holds inside chained particles called magnetosome that consists of a chain of 10-20 magnetite (Fe₃O₄) particles of single crystal having the diameter of 50-100 nm. The magnetic bacterium, holding the magnetosome, can senses geomagnetic field and recognize a direction of the magnetic line of force. The magnetic bacterium is a microaerophilic one, therefore moves along the magnetic field from an aerobic solution surface to a less aerobic surface layer of precipitate.

As disclosed in ANALYTICAL CHEMISTRY, VOL. 63, No. 3, Feb. 1, 1991 P268-P272, such bacteria can be separated to a single bacteria, and cultured in large volume. The magnetic particle in the bacterium is formed of a hexagonal rod, which diameter and shape are very uniform with a high degree of purity. It is recognized that the intensity of magnetization of a bacterium including the particles is equivalent to about 50 emu/g and its coercive force is 230 Oe with single domain structure.

Because of single domain structure having uniform direction, the particles can be collected in a narrow area of the vessel bottom when a magnet is disposed under the vessel.

The magnetic particle is covered with an organic membrane and hard to elute metal, resultantly stable with excellent dispersiveness in aqueous solution. Accordingly, it is preferable to use such bacterial magnetic particles in the nucleic acid hybridization of the invention.

As methods of extracting bacterial magnetic particles from magnetic bacteria, there has been known such methods as physically pressed crushing by a French Press, alkali boiling, enzyme treatment, ultrasonic crushing, etc. Among these methods, the ultrasonic crushing is preferably employed for obtaining large quantities of particles. After the extraction, the bacterial magnetic particles can be separated using a magnet.

In the first embodiment, a tip rack storing station 4 includes a tip rack 11 to hold disposable tips 12 to be used for dispensing washing solution and reagents. The tip rack 11 has holes, each formed to support the taper portion of the disposable tip 12, and holds the tips 12 therein before measurement.

A waste solution station 5 has a waste solution reservoir for reserving discharged waste solution that is sucked from the magnetic separation station 3 during magnetic separation/washing process.

Numeral 6 represents a washing solution station, which includes a washing solution reservoir. A plurality of washing solution stations may be provided if necessary. For instance, two washing solution stations (a first washing solution station 6a, and a second washing solution station 6b) are required if immuno reaction is performed to detect a hybridized nucleic acid.

A head station 8 has an arm unit movable in X-Z directions. The arm unit includes a mechanism for moving the head 8 with tip nozzles 10, a tip setting mechanism for tips 12 to be attached to or detached from the nozzles 10 with the movement of the head, a mechanism for the attached tips to suck or inject treatment solution (waste solution or washing solution), and a robot-hand mechanism 9 hanging from the head station 8 and being capable of holding and releasing the reaction vessel.

The hybridization apparatus may further include (H) a reagent station 7. For instance, when labeling of a nucleic acid is needed for detecting the hybridized nucleic acid, the reagent station 7 is required for reserving a labeling reagent such as alkaline phosphatase conjugated anti-DIG Fab′ fragments (anti-DIG-AP), and a chemiluminescent enzyme substrate (for example, alkaline phosphatase substrate), including, in this case, two reagent stations (a first reagent station 7a, and a second reagent station 7b).

If a previously labeled sample nucleic acid (for example, labeled by fluorescent dye) is used, the reagent station will not be needed.

The hybridization method employed in the invention can be one-step or two-step one (Sandwich method), as long as the nucleic acid in a sample hybridizes with a nucleic acid probe immobilized on the magnetic particles. The nucleic acid probe may be any one of single-stranded DNA, RNA or PNA.

### EXAMPLE 1

A description will now be given of one practical method for detecting a nucleic acid in a sample using the hybridization apparatus according to the first embodiment described above (see FIG. 1).

1. Preparation Process

(1) Production of Bacterial Magnetic Particles For production of bacterial magnetic particles, magnetic bacteria, Magnetospirillum sp. AMB-1 (Matsunaga et al. 1991), separated in a single cell were cultured in MSGM medium (Blakemore et al. 1979) (100L) anaerobically at room temperature for about 7 days. After three days of culture, 4 ml of ferric quinate solution was added per 1L of culture solution. 10,000 g of the culture was collected at 4° C. by a continuous centrifuge. The culture was suspended in 10 mM phosphate-buffered saline (PBS, ph 7.0). The bacteria were crushed under 1,500kg/cm² by a French-Press (Ohtake Mfg., 5501M), and bacterial magnetic particles were collected from the crushed bacteria, using magnetic separation with a neodymium-boron (Nd—B) magnet. The obtained magnetic particles were washed with PBS more than three times by an ultrasonic washer (Kaijo Denki Co. Ltd., CA4481) and stored at 4° C. with suspension in PBS.

(2) Synthesis of Detection Probe

By searching genus-specific regions in cyanobacterial 16S rDNA sequence to find regions having a few base difference between genera within 15-20 bases, DNA probes were designed for species-specific detection of Microcystis species. One oligonucleotide DNA was labeled with biotin at 5′ end (probe 1-biotin) out of the designed DNA probes, and the other probe was labeled with digoxigenin at 5′ end (probe 2-DIG) for detecting luminescence.

(3) Production of DNA Immobilized on Bacterial Magnetic Particles

The bacterial magnetic particles were modified using amino group on the bacterium membrane. First, 1 mg of bacterial magnetic particles (BMPs), extracted and refined from the magnetic bacteria AMB-1, were treated in 1 mL PBS containing 2.5% glutaraldehyde at room temperature for 30 minutes to introduce aldehyde group to the amino group on the membrane. After the reaction, the particles were collected magnetically and washed three times.

After the washing, 1 mg of the modified BMPs were suspended in 1 mL of PBS containing 100 μg of streptavidin (New England Bio Labs.) for 2 hours at room temperature for reaction to couple the streptavidin to the BMPs. Then, the coupled BMPs were magnetically collected and washed three times with PBS, thereafter being reduced with NaBH₄ the aldehyde group not reacted to suppress nonspecific adsorption of DNA, whereby streptavidin immobilized BMPs (SA-BMPs) were obtained. 300 μg of the SA-BMPs were applied avidin-biotin reaction with 300 pmol of oligonucleotide DNA labeled with biotin at 5′ end in 300 μl of PBS to produce oligonucleotide DNA immobilized on the bacterial magnetic particles (DNA-BMPs or probe-BMPs).

(4) Preparation of Sample DNA

Genomic DNA was extracted from cyanobacteria using modified MagExtractor-genome. All extracted genomic DNA were amplified with PCR, using primer pairs of RSF-1 and RSF-2 (antisense strand in 1523-1542 nt of E. coli) (Kawaguchi et al. 1992) for amplification of 16S rDNA in prokaryote microorganism.

When amplifying the gene, the sample nucleic acid can be labeled by PCR with the use of dUTP, which is labeled by a marker such as a fluorescent dye detectable by fluorescence, alkaline phosphatase by luminescence, ferrocene by an electro-chemical signal, or the like.

(5) Setting on Stations

\hat{1} Placed on the tip rack storing station 4 is the tip rack 11 holding tips 12, after sterilization.

\hat{2} Placed on the first washing solution station 6a is a washing solution reservoir containing the solution PBS.

\hat{3} Placed on the second washing solution station 6b is a washing solution reservoir containing detection buffer (10 mM Tris-HCl (ph 8.3), 1.5 mM MgCl₂, 50 mM KCl, and 0.1% TritonX-100).

\hat{4} Placed on the waste solution station 5 is a waste solution reservoir.

\hat{5} Placed on the first reagent station 7a is a reagent reservoir A storing 200 μl of PBS containing 0.1% BSA and 0.05% Tween 20 with a reagent a (alkaline phosphatase conjugated anti-DIG Fab′ fragments (anti-DIG-AP).

\hat{6} Placed on the second reagent station 7b is a reagent reservoir B containing a reagent b (alkaline phosphatase substrate).

Alternatively, it is possible to use as a reagent unit a probe unit in which either nucleic acid probe immobilized bacterial magnetic particles, or nucleic acid probe immobilized bacterial magnetic particles and a probe labeled with a light emitting substrate, are previously prepared and contained in a reaction vessel. This usage allows the preparation process to be outstandingly reduced and to prevent unskilled operators from lowering efficiency, which improves productivity.

2. Denature Process

100 μl of sample DNA (PCR products of 16S rDNA), 100 μg of probe-BMPs (DNA immobilized bacterial magnetic particles) and 10 pmol of probe 2-DIG are mixed and stirred in a reaction vessel (microplate, or the like). The vessel is then placed on the vessel holder in the denature station 1.

Turning on the apparatus, vessels on the denature station 1 and the annealing station 2 are heated to 95° C. and 60° C., respectively. The vessel on the denature station is first heated at 95° C. for 5 min for making the sample nucleic acid single-stranded.

3. Annealing process

After finishing the denature process, the vessel is transported onto the annealing station 2 by the arm unit. Keeping the vessel at 60° C. for 10 minutes allows the sample nucleic acid to hybridize with the probe-BMPs and the probe 2-DIG.

4. B/F Separation After the hybridization, the vessel is transported onto the vessel holder of the magnetic separation station 3 by the arm unit. Then, the magnetic force controller 20 is energized to generate magnetism, which allows collecting of the magnetic particles at the bottom of the vessel. The controller 20 is kept energizing for 3 minutes.

5. Washing Process

The arm unit transported above the tip rack 11 moves downward to attach tips 12 held on the tip rack 11 so as to fit the tip nozzles 10 to the disposable tips 12. The arm unit is transported to the magnetic separation station 3, and stops with a proper downward movement to suck the supernatant solution in the vessel by the tip nozzles 10, and then moves to the waste solution station 5 to discharge the sucked solution.

Thereafter, the arm unit is transported to the washing solution station 6 to suck the washing solution (PBS) from the first washing solution reservoir, and again transported to the magnetic separation station 3 to inject the solution into the vessel. After about 3 minutes waiting, the tip nozzles 10 again suck the supernatant solution in the vessel, and discharge this at the waste solution station 5. The magnetic force may be sometimes turned ON and OFF during waiting period. This washing operation is repeated three times.

6. Immuno-Reaction and Washing Process

The arm unit is transported onto the first reagent station 7a to suck the previously prepared 200 μl of PBS with anti-DIG-AP from the reagent reservoir, and the sucked reagent is injected into the vessel of the magnetic separation station 3. The sample is then incubated for 30 min. at room temperature. After the reaction, the supernatant solution in the vessel is sucked from the vessel and discharged at the waste solution station 5 with the movement of the arm unit.

Thereafter, the arm unit is moved to the washing solution station 6 to suck the detection buffer from the second washing solution reservoir 6b and inject it into the reaction vessel of the magnetic separation station 3 after moving back. After about 3 min. waiting, the tip nozzles 10 again suck the supernatant solution in the vessel, and discharge this at the waste solution station 5. This washing operation is repeated three times.

Dispensing 100 μl of alkaline phosphatase substrate (the reagent b), brought from the second reagent station 7b, into the vessel, this process ends.

7. Detection

The reaction vessel 13 is taking out from the apparatus, and subjected to the measurement of a light change occurred in the vessel, the light being measured by a luminescent plate reader (Lucy-2™)

As a result, the strongest light emission was observed in the PCR product sample of Microcystis aeruginosa NIES-98. That is, it was shown that sandwich hybridization method, using 16S rDNA amplified by PCR, allows specific detection of Microcystis species in cyanobacteria.

<Second Embodiment>

A second embodiment of the invention will now be explained with reference to the accompanying drawings.

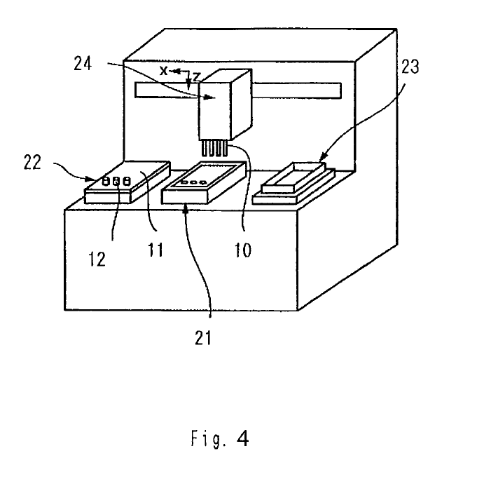

FIG. 4 is a schematic diagram showing the internal structure of an automatic nucleic acid hybridization apparatus according to the second embodiment of the invention. Those elements that are the same as corresponding elements in the first embodiment are designated by the same reference numerals and the description thereof is omitted.

Numeral 21 denotes a reaction station in which a sample nucleic acid is made single-stranded (denatured), then hybridized (annealed) with specific nucleic acid probes and then applied B/F separation. The reaction station 21 includes a vessel holder 26 for holding a reaction vessel 13, a heating-cooling device for adjusting a denaturing temperature and an annealing one, and a magnetic force controller 27.

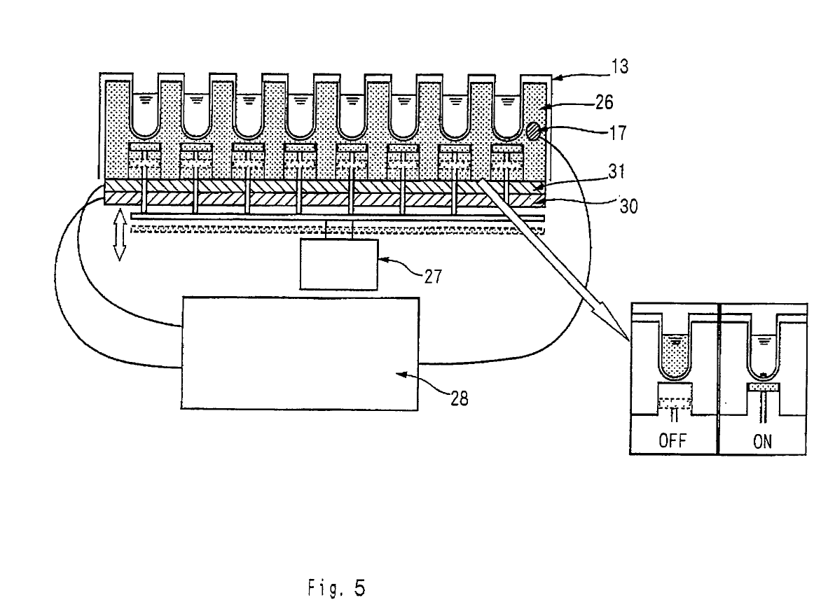

FIG. 5 is a side view showing the reaction station 21. The heating-cooling device includes a heater 30 for heating the reaction vessel holder 26, a chiller 31 for cooling it, a sensor 17 to detect the temperature, and a temperature controller 28 to control the temperature. The magnetic force controller 27 applies to each reaction vessel (well) corresponding magnetic field to act on magnetic particles in the vessel for B/F separation.

Temperature for denaturing and annealing process, and control condition for the magnetic force controller 27 are set to the same as in the first embodiment.

A tip rack/waste solution 22 includes a tip rack 11 to hold disposable tips 12 for dispensing washing solution and reagents, and a waste solution reservoir at the lower portion, where waste solution sucked from the reaction station 21 is discharged and reserved during magnetic separation/washing process. Such arrangement of the tip rack 11 and the waste solution reservoir, arranged perpendicular to a plane of the head movement, saves the apparatus space.

The tip rack 11 has holes, each formed to support the taper portion of the disposable tip 12, and holds the tips therein before measurement.

Numeral 23 represents a washing solution station, which includes a washing solution reservoir and a heating-cooling device. The washing solution is controlled to an annealing temperature by the heating-cooling device so as to prevent nonspecific adsorption or dissociation of the nucleic acid in the vessel 13 due to temperature change in the washing process.

A plurality of washing solution stations may be provided if necessary. For instance, two washing solution stations are required if immuno-detection process is requested to detect a hybridized nucleic acid.

A head station 24 has an arm unit movable in X-Z directions. The arm unit includes a mechanism for moving the head with tip nozzles 10, a tip setting mechanism for tips 12 to be attached to or detached from the respective nozzles 10 with the movement of the head, and a mechanism for the attached tips to suck or inject treatment solution (waste solution or washing solution). In this embodiment, it is not required to include a robot hand, which is provided in the first embodiment, for holding and releasing a reaction vessel.

With this structure, the apparatus not only becomes compact, but also reduces steps of moving the reaction vessel by the head when changing process, thereby reducing the loss time due to the movement.

The hybridization apparatus of this embodiment may further include a reagent station (E′) having a reagent reservoir as in the first embodiment. For instance, when labeling of a nucleic acid is needed for detecting the hybridized nucleic acid, a reagent station is required for reserving a labeling reagent, such as alkaline phosphatase conjugated anti-DIG Fab′ fragments (anti-DIG-AP), or a chemiluminescent enzyme substrate (for example, alkaline phosphatase substrate).

If a previously labeled sample nucleic acid is used, the reagent station may not be needed.

The hybridization method employed in the invention can be one-step or two-step one (Sandwich method) as in the first embodiment, and the nucleic acid probe may be any one of single-stranded DNA, RNA or PNA.

### EXAMPLE 2

A description will now be given of another practical method for detecting a nucleic acid in a sample using the hybridization apparatus according to the second embodiment described above (see FIG. 4).

1. Preparation Process

(1) Production of bacterial magnetic particles, (2) synthesis of detection probe, and (3) production of DNA immobilized on bacterial magnetic particles are the same processes as in the first embodiment, and therefore explanation will be omitted.

(4) Preparation of Sample DNA

In order to prepare a sample nucleic acid for fluorescent measurement, when amplifying the gene, 16S rDNA labeled with FITC can be synthesized by PCR using dUTP labeled with a fluorescent substance FITC.

(5) Setting on Stations

\hat{1} 100 μg of DNA probe immobilized on bacterial magnetic particles and 100 μl of labeled sample DNA are mixed and stirred in the reaction vessel, such as a micloplate, then placed on the reaction station 21 of the apparatus.

\hat{2} The tip rack 11 holding tips 12, after sterilization, is placed on the tip rack/waste solution station 22.

\hat{3} The washing solution reservoir containing washing solution is placed on the washing solution station 23.

4. Denature Process

Turning on a start switch of the apparatus, the heating-cooling device of the reaction station 21 warms up the reaction vessel to 95° C. under the control of the temperature controller 28. Keeping this temperature for 5 minutes allows the sample nucleic acid in the vessel to be made single-stranded.

5. Annealing Process

The temperature is cooled down to 60° C., and kept for 10 minutes, which hybridizes the nucleic acid probe immobilized on bacterial magnetic particles with the sample nucleic acid.

6. B/F Separation

After hybridization, the magnetic force controller 27 disposed just under the vessel is energized to generate magnetism, thereby collecting the magnetic particles at the bottom of the vessel. The controller 27 is energized for 3 minutes with the vessel kept at 60° C.

7. Washing Process

The arm unit transported to the tip rack/waste solution station 22 moves downward to attach disposable tips 12 held on the tip rack 11 to the tip nozzles 10 in fitting. The arm unit is transported to the reaction station 21, and stops with a proper downward movement to suck the supernatant solution in the vessel by the tip nozzles 10. Then, the arm unit is transported to the waste solution station 22 to discharge the sucked solution.

Thereafter, the arm unit is transported to the washing solution station 23 to suck the washing solution heated at 60° C. from the washing solution reservoir with the immersion of the nozzles 10 for a certain period of time. Then the arm unit is again transported to the reaction station 21, and injects the solution into the vessel. After 3 minutes waiting, the tip nozzles 10 again suck the supernatant solution in the vessel, and discharge this at the waste solution station 22. This washing operation is repeated three times, and finishes with the washing solution injected.

After completing the washing process, magnetic force is kept applied to the reaction vessel at 0-15° C., preferably at 4° C., whereby the magnetic particles in the solution are kept cohered at the bottom of the vessel. This prevents the change of characteristic of the solution. At this time, the heating-cooling device of the washing solution station is disabled. In addition, if the reaction vessel 13 has a cover to shield light from the outside of the apparatus, the fluorescent substance captured in the nucleic acid is prevented from its deterioration.

8. Detection

The reaction vessel 13 is taken out from the apparatus, and subjected to measurement of a light change occurred in the vessel, the light being measured by a fluorescent plate reader (FLUOstar™).

As a result, the strongest light emission was observed in the PCR product sample of Microcystis aeruginosa NIES-98.

What is claimed is:

1. An automatic nucleic acid hybridization apparatus comprising:
(A) a denature station having a reaction vessel holder and a heating-cooling device;
(B) an annealing station having a reaction vessel holder and a heating-cooling device;
(C) a magnetic separation station having a reaction vessel holder and a magnetic force controller;
(D) a tip rack storing station having a tip rack;
(E) a washing solution station having a washing solution reservoir;
(F) a waste solution station having a waste solution reservoir; and
(G) a head station having an arm unit movable in X-Z directions, the arm unit including a tip setting mechanism having a plurality of tip nozzles for respective tips to be attached to or detached from the nozzles, a mechanism for the attached tips to suck and/or inject treatment solution, and a robot-hand mechanism capable of holding and releasing a reaction vessel.

2. The apparatus as claimed in claim 1, wherein the waste solution station (F) is disposed at a lower portion of the tip rack storing station (D).

3. The apparatus as claimed in claim 2, further comprising:
(H) a reagent station having a reagent reservoir.

4. A method for detecting a nucleic acid in a sample using the hybridization apparatus as claimed in claim 3, the method automatically executing the following steps(1)-(10) and further (11)-(15) if necessary, and thereafter measuring the amount of labeled nucleic acid in the reaction vessel:
(1) setting on the denature station a reaction vessel in which a nucleic acid probe immobilized on magnetic particles, a labeled probe and a sample nucleic acid, or a nucleic acid probe immobilized on magnetic particles and a labeled sample nucleic acid are injected and mixed, setting the temperature inside the vessel to a denaturing temperature of the nucleic acid by the heating-cooling device, and making the sample nucleic acid single-stranded with the temperature kept for a certain period of time;
(2) transporting the reaction vessel on the denature station to the annealing station with actuation of the arm unit;
(3) annealing the nucleic acid by setting the temperature inside the vessel to an annealing temperature by the heating-cooling device with the temperature kept for a certain period of time;
(4) transporting the reaction vessel from the annealing station to the magnetic separation station by the arm unit;
(5) biasing the nucleic acid bound with the magnetic particles in the vessel with the magnetic force controller energized;
(6) transporting the arm unit to the tip rack storing station, and attaching tips to respective tip nozzles;
(7) transporting the arm unit to the magnetic separation station, and sucking supernatant solution in the reaction vessel by the tip nozzles;
(8) transporting the arm unit to the waste solution station, and discharging the sucked supernatant solution into the waste solution reservoir;
(9) transporting the arm unit to the washing solution station, sucking the washing solution from the washing solution reservoir, and dispensing the washing solution into the reaction vessel on the magnetic separation station;
(10) repeating the washing operation specified insteps (7)-(9) by given times;
(11) after finishing steps (7)-(8), transporting the arm unit to a first reagent station, sucking a marking reagent from a reagent reservoir, dispensing the reagent into the reaction vessel on the magnetic separation station, and leaving still for a certain period of time;
(12) sucking the supernatant solution in the reaction vessel by the tip nozzles, transporting the arm unit to the waste solution station, and discharging the sucked supernatant in the tip nozzles into the waste solution reservoir;
(13) transporting the arm unit to a second washing solution station, sucking washing solution from a second washing solution reservoir, dispensing it into the reaction vessel on the magnetic separation station, and leaving still for a certain period of time;
(14) repeating the washing operation specified in steps
(12)-(13) by given times, and executing step (12); and
(15) transporting the arm unit to a second reagent station, sucking a color developing agent from a reagent reservoir, and dispensing it into the reaction vessel on the magnetic separation station.

5. The method as claimed in claim 4, wherein a reagent unit having previously prepared nucleic acid probe immobilized on magnetic particles is used as the reaction vessel.

6. The method as claimed in claim 4, wherein a reagent unit, having previously prepared nucleic acid probe immobilized on magnetic particles and a luminescence detection probe, is used as the reaction vessel.

7. The method as claimed in claim 4, wherein the magnetic particles are bacterial magnetic particles.

8. The method as claimed in claim 4, wherein the nucleic acid immobilized on the magnetic particles is a single-stranded DNA, RNA, or PNA.

9. The method as claimed in claim 4, wherein the sample nucleic acid is labeled with fluorescent dyes, alkaline phosphatase, or ferrocene.

10. An automatic nucleic acid hybridization apparatus, comprising:
(A′) a reaction station having a reaction vessel holder, a heating-cooling device, and a magnetic force controller;
(B′) a tip rack/waste solution station having a tip rack and a waste solution reservoir;
(C′) a washing solution station having a washing solution reservoir and a heating-cooling device; and
(D′) a head station having an arm unit movable in X-Z directions, the arm unit comprising a tip setting mechanism having a plurality of tip nozzles for respective tips to be attached to or detached from, and a mechanism for the attached tips to suck and/or inject treatment solution.

11. The apparatus as claimed in claim 10, further comprising (E′) a reagent station having a reagent reservoir.

12. A method for detecting a nucleic acid in a sample using the hybridization apparatus as claimed in claim 10, the method automatically executing the following steps (1′)-(8′), and thereafter measuring the amount of labeled nucleic acid in the reaction vessel:
(1′) setting on the reaction station a reaction vessel in which a nucleic acid probe immobilized on magnetic particles and a labeled sample nucleic acid are injected and mixed, setting the temperature inside the vessel to a denaturing temperature of the nucleic acid by the heating-cooling device, and making the sample nucleic acid single-stranded with the temperature maintained for a certain period of time;
(2′) annealing the nucleic acid by changing the temperature inside the vessel to an annealing temperature with the annealing temperature maintained for a certain period of time;
(3′) biasing in the vessel the nucleic acid bound with the magnetic particles with the magnetic force controller enabled (B/F separation);
(3′) transporting the arm unit to the tip rack/waste solution station, and attaching tips to respective tip nozzles;
(5′) transporting the arm unit to the reaction station, and sucking supernatant solution in the reaction vessel by the tip nozzles;
(6′) transporting the arm unit to the tip rack/waste solution station, and discharging the sucked supernatant solution into the waste solution reservoir;
(7′) transporting the arm unit to the washing solution station, sucking from the washing solution reservoir the washing solution previously adjusted to the annealing temperature by the heating-cooling device, and dispensing the washing solution into the reaction vessel of the reaction station after immersion of the tip nozzles in the washing solution for a certain period of time; and
(8′) repeating the washing operation specified at steps (5′)-(7′) by given times.

13. The method as claimed in claim 12, wherein the temperature of the washing solution is adjusted either during the term of running the apparatus or at any one of steps (1′)-(6′).

14. The method as claimed in claims 13, wherein the tip nozzles maintain the same temperature as the annealing one through the washing solution by immersing the tip nozzles in the washing solution in the washing solution reservoir when the arm unit is in a standby state before the washing steps or during the washing steps.

15. The method as claimed in claim 14, wherein the reaction vessel maintains the inside temperature at 0-15° C. after finishing step (8′).

16. The method as claimed in claim 12, wherein, in B/F separation step (step 3′), the magnetic force is controlled so as to make the magnetic particles collected and immovable at the bottom only of the reaction vessel.

17. The method as claimed in claim 16, wherein the B/F separation and washing steps after finishing hybridization reaction comprise the steps of:
sucking and discharging the supernatant solution with the magnetic force controlled to make the magnetic particles immovable at the bottom only of the reaction vessel; and
injecting into the reaction vessel the washing solution under the immovable state of the magnetic particles, or after making the magnetic particles movable with the control of the magnetic force.

18. The method as claimed in claim 17, wherein the B/F separation and washing steps further comprises moving the magnetic particles in the washing solution with the repetition of turning ON and OFF of the magnetic force.

19. The method as claimed in claim 18, wherein the turning ON and OFF of the magnetic force is implemented by rotating magnets about 180 degrees.
